(19) United States
(12) Patent Application Publication
(10) Pub. No.: US 2010/0094039 A1
(43) Pub. Date: Apr. 15, 2010

(54) PROCESS FOR PREPARING DIARYL CARBONATES

(75) Inventor: Pieter Ooms, Krefeld (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/575,040

(22) Filed: Oct. 7, 2009

(30) Foreign Application Priority Data
Oct. 8, 2008 (DE) 102008050828.4

(51) Int. Cl. C07C 69/96; C07C 68/02

(52) U.S. Cl. 558/274

(57) ABSTRACT

The invention relates to a process for preparing diaryl carbonates by reacting monophenols with phosgene or aryl chlorocarbonates with elimination of hydrogen chloride in the presence of mixed hydroxides of elements from groups 2-14 of the periodic table (IUPAC, new) as heterogeneous catalysts.

### RELATED APPLICATIONS

This application claims benefit to German Patent Application No. 10 2008 050 828.4, filed Oct. 8, 2008, which is incorporated herein by reference in its entirety for all useful purposes.

### BACKGROUND OF THE INVENTION

The invention relates to a process for preparing diaryl carbonates by reacting aromatic monohydroxy) compounds with phosgene or aryl chlorocarbonates with elimination of hydrogen chloride in the presence of mixed hydroxides of elements from groups 2-14 of the periodic table (IUPAC, new) as heterogeneous catalysts.

Diaryl carbonates are suitable for preparing polycarbonates by the melt transesterification process (see, for example, in Chemistry and Physics of Polycarbonates, Polymer Reviews, H. Schnell, Vol. 9, John Wiley and Sons, Inc. (1964)) or for preparing phenylurethanes, or are precursors of active ingredients from the pharmaceuticals and crop protection sector.

It is known that diaryl carbonates can be obtained by phase interface phosgenation (Schotten-Baumann reaction) of aromatic hydroxyl compounds. In this method, the use of solvents and sodium hydroxide solution has an adverse effect, since the aqueous alkali can result in partial hydrolysis of phosgene or chlorocarbonic ester. In each case, large amounts of sodium chloride are obtained as a by-product. Moreover, it is necessary to take care that the solvent is recovered.

A condensation has therefore been proposed without use of solvents and sodium hydroxide solution in the presence of tetramethylammonium halides as catalysts (U.S. Pat. No. 2,837,555). Here, though, the amounts of catalyst required are relatively large. It is generally necessary to work with 5 to 7% by weight of catalyst, based on the amount of phenol used, in order to obtain economically viable reaction rates; the reaction temperatures of 180° C. to 215° C. additionally entail the risk of decomposition of the thermally labile tetramethylammonium halides. The catalyst additionally has to be removed by washing with water, which considerably complicates its recovery. Furthermore, far more than the stoichiometrically necessary amount of phosgene is consumed.

In a further method (U.S. Pat. No. 3,234,263), diaryl carbonates are obtained by heating aryl chlorocarbonates in the presence of large amounts of alkali metal/alkaline earth metal compounds with tertiary nitrogen bases as catalysts. However, this process has the disadvantage that high temperatures have to be employed and the catalysts such as alkali metal/alkaline earth metal compounds have to be partly dissolved, in order to arrive at even remotely acceptable reaction times. In this process, half of the phosgene originally used is lost in the form of CO₂. Moreover, the chlorocarbonic ester has to be synthesized in a preceding separate process step.

According to U.S. Pat. No. 2,362,865, diaryl carbonates are obtained by phosgenating monophenols in the presence of metallic titanium, iron, zinc and tin, or in the form of soluble salts thereof, particularly of the chlorides and phenoxides. Even though very good yields are obtained, it is difficult to separate the catalysts from the products. Even in the case of distillations, a certain volatility of these compounds and also thermal decompositions by these compounds have to be expected, which lead to contamination, reduction in quality and yield losses.

It thus appears to be advisable to use heterogeneous insoluble catalysts, which substantially simplify workup of the reaction mixture. Proposals to this end have also been made. For instance, the teaching of EP-A-516 355 recommends aluminium trifluoride in particular, which is optionally applied to supports such as aluminosilicates. The synthesis of aluminium fluoride is, however, very complicated and expensive due to the handling of fluorine or hydrofluoric acid. Moreover, WO 91/06526 describes metal salts on porous supports as catalysts for the inventive conversions. As is evident from the experimental examples, fully continuous phosgenation of phenol over such catalysts is possible only in the gas phase, which, though, entails relatively high reaction temperatures and the risk of decomposition of the sensitive phenyl chloroformate. It is obviously impossible to perform phosgenation of phenol with these catalysts in the liquid phase, since the hot liquid phenol washes out the active catalyst constituents.

It was thus an object of the present invention to develop easily obtainable, effective heterogeneous catalysts.

It has now been found that mixed hydroxides of elements from groups 2-14 of the periodic table (IUPAC, new), for example hydrotalcite, are suitable catalysts for the reaction of phosgene or aryl chlorocarbonates with monophenols to give diaryl carbonates, the hydrogen chloride formed being reusable as a reactant in other processes or by oxidation to chlorine.

The process according to the invention has the great advantage of achieving very high selectivities and good phenol conversions, in order thus to arrive at a product with high purity. Furthermore, the catalyst can be removed very readily, thus substantially easing the workup.

### EMBODIMENTS OF THE INVENTION

An embodiment of the present invention is process for preparing a diaryl carbonate comprising reacting a monophenol with phosgene or an aryl chlorocarbonate, wherein said reaction is performed in the presence of a compound of general formula (III)

[M(II)₁₋ₓ M(III)ₓ M(IV)_{y} (OH)₂] Aⁿ⁻_{z/n}·m H₂O (III)

wherein
M(II) is a divalent metal cation;
M(III) is a trivalent metal cation;
M(IV) is a tetravalent metal cation;
x is a number from 0.1 to 0.5;
y is a number from 0 to 0.5;
z is 1+y;
m is an integer from 0 to 32;
A is an anion; and
n is 1 or 2
   as a heterogeneous catalyst.

Another embodiment of the present invention is the above process, wherein said anion is selected from the group consisting of CO₃²⁻, OH⁻, SO₄²⁻, NO₃⁻, CrO₄²⁻, and Cl⁻.

Another embodiment of the present invention is the above process, wherein said reaction is performed at a temperature in the range of from 50 to 450° C. and at a pressure in the range of from 0.05 to 20 bar.

Another embodiment of the present invention is the above process, wherein said heterogeneous catalyst has a surface area, as determined by the BET method, of from 0.1 to 400 m²/g and is used in an amount of from 0.5 to 100% by weight, based on the amount of said monophenol, in not fully continuous mode, or with a space velocity of from 0.1 to 20 g of monophenol per g of catalyst per hour in fully continuous mode.

Another embodiment of the present invention is the above process, wherein said divalent metal cation M(II) is Mg, Ni, or Zn, said trivalent metal cation M(III) is Al, and said tetravalent metal cation M(IV) is Ti or Zr.

Another embodiment of the present invention is the above process, wherein said diaryl carbonate is prepared continuously.

Another embodiment of the present invention is the above process, wherein said process is conducted at a temperature in the range of from 100 to 350° C. and at a pressure in the range of from 0.05 to 20 bar.

Another embodiment of the present invention is the above process, wherein said reaction is effected in the gas phase.

Another embodiment of the present invention is the above process, wherein said reaction is effected in countercurrent in the trickle phase.

Another embodiment of the present invention is the above process, wherein said heterogenous catalyst consists of a supported active phase of the compound of general formula (III).

Yet another embodiment of the present invention is a diaryl carbonate obtained by the above process.

### DESCRIPTION OF THE INVENTION

The present invention accordingly provides a process for preparing diaryl carbonates by reacting monophenols with phosgene or aryl chloroformates, which is characterized in that it works in the presence of mixed hydroxides of elements from groups 2-14 of the periodic table (IUPAC, new) as heterogeneous catalysts.

Monophenols for the process according to the invention are those of the formula

Ar—OH (I)

in which
Ar is phenyl, naphthyl, anthryl, phenanthryl, indanyl, tetrahydronaphthyl or the radical of a 5- or 6-membered aromatic heterocycle with 1 or 2 heteroatoms from the group of N, O and S, where these isocyclic and heterocyclic radicals may be substituted by 1 or 2 substituents such as straight-chain or branched C₁-C₄-alkyl, straight-chain or branched C₁-C₄-alkoxy, straight-chain or branched C₁-C₄-alkoxycarbonyl, which may be substituted by phenyl, cyano and halogen (e.g. F, Cl, Br) and where, in addition, the heterocyclic radicals may be joined to a fused-on benzene ring.

Examples of monophenols of the formula (I) are: phenol, o-, m- and p-cresol, o-, m- and p-isopropylphenol, the corresponding halo- or alkoxyphenols, such as p-chlorophenol or p-methoxyphenol, methyl salicylate, ethyl salicylate, and also monohydroxyl compounds of naphthalene, of anthracene and of phenanthrene, and additionally 4-hydroxypyridine and hydroxyquinolines. Preference is given to using phenol and optionally substituted phenols, very particular preference to using phenol itself.

The process according to the invention can be performed either with phosgene or with aryl chlorocarbonates. In the case of performance with phosgene, the aryl chlorocarbonate is formed first, which is reacted with further monophenol present in the reaction mixture to give diaryl carbonate.

When the starting materials are aryl chlorocarbonates and a monophenol, symmetric or unsymmetric diaryl carbonates can be obtained.

Suitable aryl chlorocarbonates for the process according to the invention are those of the formula (II)

Ar—OCOCl (II)

in which Ar is as defined in formula (I).

Suitable mixed hydroxides in the context of the invention are compounds of the general formula (III)

[M(II)₁₋ₓ M(III)ₓ M(IV)_{y} (OH)₂]Aⁿ⁻_{z/n}·m H₂O (III)

in which
M (II) is a divalent metal cation and
M (III) is a trivalent metal cation and
M(IV) is a tetravalent metal cation and
x is from 0.1 to 0.5 and
y is from 0 to 0.5
z is 1+y and
m is from 0 to 32
A is an anion such as CO₃²⁻, OH⁻, SO₄²⁻, NO₃⁻, CrO₄²⁻ or Cl⁻, preferably CO₃²⁻, OH⁻, SO₄²⁻
n is 1 or 2.

Examples of metal cations M(II) include:
divalent metal cations such as Be, Mg, Ca, Zn, Fe, Mn, Co, Ni, Cu, Cd, preference being given to Mg, Ni, Zn and Fe, particular preference to Mg, Ni and Zn.

Examples of metal cations M(III) include:
trivalent metal cations such as Al, Ga, Ni, Co, Fe, Mn, Al, Cr, Fe, Sn, V, preference being given to Al, Cr, Fe, particular preference to Al.

Examples of metal cations M(IV) include: tetravalent metal cations such as Ti, Zr and Hf, preference being given to Ti and Zr, particular preference to Ti.

In the mixed hydroxides, it is also possible for a plurality of different metal cations M(II) or metal cations M(III), or else metal cations M(II) or metal cations M(III) of the same element in different valency, to occur alongside one another.

The mixed hydroxides used in accordance with the invention may possess a layer structure composed of polycations and -anions, for example hydrotalcite, or a different structure, for example ettringite.

Useful mixed hydroxides are both those from natural sources, i.e. various minerals, for example
hydrotalcite Mg₆Al₂(OH)₁₆CO₃·4H₂O
manasseite Mg₆Al₂(OH)₁₆CO₃·4H₂O
pyroaurite Mg₆Fe₂(OH)₁₆CO₃·4.5H₂O
sjörgrenite Mg₆Fe₂(OH)₁₆CO₃·4.5H₂O
stichtite Mg₆Cr₂(OH)₁₆CO₃·4H₂O
barbertonite Mg₆Cr₂(OH)₁₆CO₃·4H₂O
takovite Ni₆Al₂(OH)₁₆CO₃·OH·4H₂O
reevesite Ni₆Fe₂(OH)₁₆CO₃·4H₂O
desautelsite Mg₆Mn₂(OH)₁₆CO₃·4H₂O
hydrocalumite [Ca₂Al(OH)₆]OH·6H₂O
magaldrate [Mg₁₀Al₅(OH)₃₁](SO₄)₂·mH₂O
ettringite [Ca₆Al₂(OH)₁₂](SO₄)₃·26H₂O,
   and synthetic mixed hydroxides, generally prepared by precipitation from solutions of precursors, for example metal salts or metal oxides, and bases.

Such mixed hydroxides and their origin or preparation processes for such compounds are described, for example, in Clays and Clay Minerals 25 (1977) 14, 23(1975) 369, Catalysis Today 11 (1991) 173, Chimia 24 (1970) 99, and EP-A 749 941, EP-A 421 677, EP-A 684 872, EP-A 0749941, DE-A 2 024 281 and WO 95/17246.

Particularly suitable heterogeneous catalysts are mixed hydroxides with hydrotalcite structure, for example mixed hydroxides of magnesium, zinc, nickel, aluminium, cobalt, tin and titanium.

The mixed hydroxides in the context of the invention may be present in crystalline form in various polymorphs. They may be entirely or partly amorphous and be dried or partly dried or be used as hydrates.

Reaction of mixed metal salts in the presence of bases at temperatures of 80 to 100° C. first forms hydroxycarbonates, which are converted to the anhydrous mixed hydroxides at relatively high calcination temperatures with decarboxylation and with progressive dewatering. For instance, in the event of calcination above 500° C., hydrotalcite Mg₆Al₂(OH)₁₆CO₃·4H₂O is converted to Mg₆Al₂O₅(OH)₂. According to the type of starting hydroxide or hydroxide carbonate, it is possible for the calcination to pass through various of the abovementioned polymorphs of the mixed hydroxide.

Preferred mixed hydroxides possess BET surface areas of 0.1 to 500 m²/g, more preferably those of 0.5 to 450 m²/g and most preferably those of 1 to 300 m²/g.

The catalysts can be used, for example, in the form of powder or shaped bodies, and be removed again after the reaction, for example by filtration, sedimentation or centrifugation. In the case of arrangement as a fixed bed, the metallates are preferably used in the form of shaped bodies, for example as spheres, cylinders, rods, hollow cylinders, rings etc.

When working with suspended catalyst, the mixed hydroxide catalysts are used in stirred vessels or bubble columns in amounts of 0.5 to 100% by weight, preferably of 5 to 100% by weight and more preferably of 5 to 50% by weight, based on the amount of monophenol used.

In the case of continuous mode in countercurrent or cocurrent or in the trickle phase or in the gas phase over a fixed bed catalyst, catalyst hourly space velocities of 0.1 to 20 g of monophenol per g of catalyst per hour, preferably 0.2 to 10 g·g⁻¹·h⁻¹ and more preferably of 0.2 to 5 g·g⁻¹·h⁻¹ are used.

The mixed hydroxides used in batehwise experiments, given the same feedstocks, can be used repeatedly without purification. In the case of a change in the feedstocks, the mixed hydroxides are appropriately purified by extracting with inert solvents, as specified, for example, further down as reaction media, or with alcohols such as methanol, ethanol, isopropanol or butanol, with esters or amides of acetic acid, or by treatment with superheated steam or air.

In continuous mode, the mixed hydroxides used can remain in the reactor over a long period. A regeneration can, if appropriate, be effected, for example, by passing over superheated steam, if appropriate with addition of minor amounts of air (for instance 0.1 to 20% by weight, based on the amount of steam used) at 150 to 800° C. or by passing over 0.01 to 20% by weight of oxygen-containing diluent gases such as nitrogen or carbon dioxide, or by means of carbon dioxide alone at 200 to 800° C. The preferred regeneration temperature is 150 to 700° C., more preferably 200 to 600° C.

The process according to the invention is performed at a temperature in the range from 50 to 450° C., preferably 100 to 400° C., more preferably 100 to 350° C. During the performance of the process according to the invention, the temperature can be varied within the range specified, preferably increased.

The process according to the invention is performed at a pressure of 0.05 to 20 bar, preferably 1 to 5 bar.

The process according to the invention can optionally be performed using solvents such as aliphatic and aromatic hydrocarbons, e.g. hexane, octane, benzene, isomeric xylenes, diethylbenzene, alkylnaphthalenes, biphenyl or halogenated hydrocarbons such as dichloromethane and trichloroethylene.

The process according to the invention can be performed either in the gas phase or in the liquid phase.

The process is preferably performed in the melt, for example by introducing phosgene or an aryl chlorocarbonate of the formula (II) into a suspension of a mixed hydroxide in a melt of the monophenol of the formula (I) and, after the reaction has ended, removing the catalyst, for example by filtration or centrifugation.

The process is performed in the gas phase by evaporating phosgene and monophenol, and passing the mixture over a bed of a catalyst in piece form arranged in a tube.

A further preferred embodiment of the synthesis is the sparging of a melt of the monophenol of the formula (I), with mixed hydroxide catalyst suspended therein, with phosgene or phosgene-hydrogen chloride mixtures or with aryl chlorocarbonates of the formula (H) in a continuous bubble column or bubble column cascade.

A further preferred embodiment is the cocurrent method, in which monophenols of the formula (I) and phosgene or aryl chlorocarbonate of the formula (II) are applied in cocurrent, for example from the top, to a catalyst bed arranged in a tube, and hydrogen chloride and phosgenation products are drawn off at the bottom of the tube.

A further preferred embodiment is the performance of the inventive reaction in countercurrent in the trickle phase, in which ease the monophenol of the formula (I) is introduced as a melt or in the form of a solution to the top of a bed of mixed hydroxide, and a stream of phosgene or aryl chlorocarbonate is sent counter to this liquid stream from below. Appropriately, this embodiment is performed in a vertical crude reactor, which may also contain intermediate trays for better distribution of gas and liquid flow.

A further preferred embodiment is the gas phase method at temperatures of 150 to 450° C., preferably 200 to 350° C., with pressures of 0.05 to 20, preferably 0.1 to 4 bar, more preferably 0.1 to 3 bar.

In this process, the pressure is varied with the temperature such that the components remain in the gas phase and do not condense on the catalyst bed.

The molar ratio of the monophenol reactant of the formula (I) to the phosgene reactant is 0.5 to 8:1, preferably 1.5 to 3:1. The equivalent molar ratio in this case is 2:1.

In a corresponding manner, the monophenol is reacted with an aryl chlorocarbonate in a molar ratio of 0.25 to 4:1, preferably 0.8 to 1.5:1. In this case, the molar ratio is 1:1.

The crude diaryl carbonate obtained in accordance with the invention by heterogeneous catalysis is frequently already very pure and can, after degassing to remove residual hydrogen chloride or other volatile substances, be used for many purposes actually in this form. For more demanding applications, the diaryl carbonate can optionally be purified further by known methods, for example by distillation or crystallization.

The invention further provides a process for preparing diaryl carbonates using supported catalysts. Suitable heterogeneous catalysts in this case are especially compounds of the formula (III)

[M(II)₁₋ₓ M(III)ₓ M(IV)_{y} (OH)₂]Aⁿ⁻_{z/n}·m H₂O (III)

on support materials, which may also be doped.

The compounds of the formula (III) can also be mixed with further substances as a constituent of a catalyst formulation, in order possibly to generate synergistic effects. Suitable examples for this purpose are silicon dioxide, graphite, titanium dioxide with ruffle or anatase structure, zirconium dioxide, aluminium oxide, silicon carbides or mixtures thereof, preferably titanium dioxide, zirconium dioxide, aluminium oxide or mixtures thereof.

The reaction to give the diaryl carbonate can be performed in a plurality of stages. It can be performed batchwise, preferably continuously as a fluidized bed or fixed bed method, preferably as a fixed bed method, more preferably in tube bundle reactors over the heterogeneous catalysts.

A preferred embodiment consists in using a structured catalyst bed in which the catalyst activity rises in flow direction. Such structuring of the catalyst bed can be effected by different impregnation of the catalyst supports with active material or by different dilution of the catalyst with inert material.

The heat of reaction can be utilized in an advantageous manner to raise high-pressure steam.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

The catalysts used are commercially available products or were prepared by known methods (see Catalysis Today 11 (1991) 173, EP-A 421 677, EP-A 749 941, WO 95/17248, EP-A 684 872, DE-A 2 024 282).

### Example 1

In a flat-flange pot with baffles, a sparging stirrer and reflux condenser, 141 g (1.50 mol) of phenol were sparged continuously in the presence of 14.1 g (10% by weight based on phenol) of a pulverulent hydrotalcite (molar Mg/Al ratio=2:1) at 140° C. with 0.75 mol/h of phosgene. After about 2 h of reaction time, the phenol conversion was 29.8%, and only diphenyl carbonate (57.6 g) had formed. The selectivity for the carbonate was >99.7%.

### Example 2

Example 1 was repeated with 14.1 g of a pulverulent zinc aluminium hydroxide (molar Zn/Al ratio=2:1) at 140° C. After 2 h of reaction time, the phenol conversion was 15.2%, and 0.03 g of phenyl chloroformate and 23.9 g of diphenyl carbonate had formed. The selectivity for the carbonate was approx. 90%.

### Example 3

Example 1 was repeated with 14.1 g of a pulverulent nickel(II) aluminium hydroxide (molar Ni/Al ratio=2:1) at 140° C. After 2 h of reaction time, the phenol conversion was 11.2%, and 0.6 g of phenyl chloroformate and 17.4 g of diphenyl carbonate had formed. The selectivity for the carbonate was approx. 99%.

### Example 4

Example 1 was repeated with 14.1 g of a pulverulent hydrotalcite (molar Mg/Al ratio 7:3) from Condea at 140° C. After 2 h of reaction time, the phenol conversion was 24.4%, and 39.0 g of diphenyl carbonate had formed. The carbonate selectivity was >99%.

### Example 5

Example 1 was repeated with 14.1 g of a pulverulent magnesium tin(II) hydroxide (molar Mg/Sn ratio=1.0/0.034) at 140° C. After 2 h of reaction time, the phenol conversion was 24.6%, and 39.2 g of diphenyl carbonate had formed. The selectivity for the carbonate was greater than 99%.

### Example 6

Example 1 was repeated with 14.1 g of a pulverulent magnesium titanium(IV) hydroxide (molar Mg/Ti ratio=1.0/0.050) at 140° C. After 2 h of reaction time, the phenol conversion was 26.6%, and 42.4 g of diphenyl carbonate had formed. The carbonate selectivity was >99%.

### Example 7

Example 1 was repeated with 14.1 g of a pulverulent hydrotalcite (molar ratio=Mg/Al 7:3) from Condea at 140° C. After 2 h of reaction time, the phenol conversion was 24.4%, and 39.0 g of diphenyl carbonate had formed. The carbonate selectivity was >99%.

### Example 8

Example 1 was repeated with 14.1 g of a pulverulent nickel(II) magnesium aluminium hydroxide (molar Ni/Mg/Al ratio=0.14/2.34/1.0) at 140° C. After 2 h of reaction time, the phenol conversion was 19.9%, and 31.0 g of diphenyl carbonate had formed. The selectivity for the carbonate was approx. 97%.

### Example 9

Example 1 was repeated with 1.41 g of a pulverulent titanium(IV) magnesium aluminium hydroxide (molar Ti/Mg/Al ratio=0.26/2.63/1.0) at 140° C. After 2 h of reaction time, the phenol conversion was 22.8%, and 36.5 g of diphenyl carbonate were formed. The selectivity for the carbonate was >99%.

### Example 10

In a three-neck flask with thermometer and reflux condenser, a mixture of 9.4 g (0.10 mol) of phenol and 15.7 g (0.10 mol) of phenyl chloroformate was heated to 140° C. in the presence of 0.94 g (10% by weight based on phenol) of a pulverulent hydrotalcite (molar ratio=Mg/Al2:1). After 5 h of reaction time, 90.7% of the phenol had been converted to diphenyl carbonate.

### Example 11

Example 10 was repeated with 0.94 g of a pulverulent zinc aluminium hydroxide (molar ratio=2:1) at 140° C. After 1 h of reaction time, the phenol conversion to diphenyl carbonate was 99.8%. The carbonate selectivity was >99%.

### Example 12

Example 10 was repeated with 0.94 g of nickel(II) aluminium hydroxide (molar ratio=2:1) at 140° C. After 3 h of reaction time, the phenol conversion to diphenyl carbonate was 97.5%. The carbonate selectivity was >99%.

### Example 13

Example 10 was repeated with 0.94 g of a pulverulent magnesium tin hydroxide (molar ratio=1.0/0.034) at 140° C. After 2 h of reaction time, the phenol conversion to diphenyl carbonate was 49.7%. The selectivity for the carbonate was >99%.

### Example 14

Example 10 was repeated with 0.94 g of a pulverulent magnesium titanium(IV) hydroxide (molar ratio=1.0/0.050) at 140° C. After 2 h of reaction time, the phenol conversion to diphenyl carbonate was 86.1%. The carbonate selectivity was >99%.

### Example 15

Example 10 was repeated with 0.94 g of a pulverulent hydrotalcite (molar ratio=Mg/Al 7:3) from Condea at 140° C. After 1 h of reaction time, the phenol conversion to diphenyl carbonate was 98.8%. The selectivity for the carbonate was >99%.

### Example 16

Example 10 was repeated with 0.94 g of a pulverulent titanium(IV) magnesium aluminium hydroxide (molar ratio=0.26/2.63/1.0). After 1 h of reaction time, the phenol conversion to diphenyl carbonate was 98.6%. The selectivity for the carbonate was >99%.

### Comparative Example 1

Example 1 was repeated without addition of mixed hydroxide at 140° C. After 2 h of reaction time, the phenol conversion was less than 0.2%.

### Comparative Example 2

Example 1 was repeated in the presence of pulverulent aluminium oxide 507-C-I at 140° C. After 2 h of reaction time, the phenol conversion was 41% and the carbonate selectivity was >99.5%.

### Comparative Example 3

Example 11 was repeated in the presence of pulverulent aluminium oxide 507-C-I at 140° C. After 2 h of reaction time, the phenol conversion was 90% and the selectivity for the carbonate was >99%.

1. A process for preparing a diaryl carbonate comprising reacting a monophenol with phosgene or an aryl chlorocarbonate, wherein said reaction is performed in the presence of a compound of general formula (III)
[M(II)₁₋ₓ M(III)ₓ M(IV)_{y} (OH)₂] Aⁿ⁻_{z/n}·m H₂O (III)
wherein
M(II) is a divalent metal cation;
M(III) is a trivalent metal cation;
M(IV) is a tetravalent metal cation;
x is a number from 0.1 to 0.5;
y is a number from 0 to 0.5;
z is 1+y;
m is an integer from 0 to 32;
A is an anion; and
n is 1 or 2
as a heterogeneous catalyst.

2. The process of claim 1, wherein said anion is selected from the group consisting of CO₃²⁻, OH⁻, SO₄²⁻, NO₃⁻, CrO₄²⁻, and Cl⁻.

3. The process of claim 1, wherein said reaction is performed at a temperature in the range of from 50 to 450° C. and at a pressure in the range of from 0.05 to 20 bar.

4. The process of claim 1, wherein said heterogeneous catalyst has a surface area, as determined by the BET method, of from 0.1 to 400 m²/g and is used in an amount of from 0.5 to 100% by weight, based on the amount of said monophenol, in not fully continuous mode, or with a space velocity of from 0.1 to 20 g of monophenol per g of catalyst per hour in fully continuous mode.

5. The process of claim 1, wherein said divalent metal cation M(II) is Mg, Ni, or Zn, said trivalent metal cation M(III) is Al, and said tetravalent metal cation M(IV) is Ti or Zr.

6. The process of claim 1, wherein said diaryl carbonate is prepared continuously.

7. The process of claim 1, wherein said process is conducted at a temperature in the range of from 100 to 350° C. and at a pressure in the range of from 0.05 to 20 bar.

8. The process of claim 1, wherein said reaction is effected in the gas phase.

9. The process of claim 1, wherein said reaction is effected in countercurrent in the trickle phase.

10. The process of claim 1, wherein said heterogenous catalyst consists of a supported active phase of the compound of general formula (III).

11. A diaryl carbonate obtained by the process of claim 1.
